(19) United States
(12) Patent Application Publication
(10) Pub. No.: US 2011/0275098 A1
(43) Pub. Date: Nov. 10, 2011

(54) Method for Evaluating The Immunogenicity of Proteins

(76) Inventors: Bernard Maillere, Versailles (FR); Stéphanie Delluc-Desroches, La Chapelle d'Aunainville (FR)

(21) Appl. No.: 13/139,570

(22) PCT Filed: Dec. 15, 2009
(86) PCT No.: PCT/FR09/01424

§ 371 Date: Jul. 1, 2011

(30) Foreign Application Priority Data
Dec. 18, 2008 (FR) 0807123

(51) Int. Cl. C12Q 1/02; G01N 33/566

(52) U.S. Cl. 435/7.92; 435/29

(57) ABSTRACT

The invention relates to a method for evaluating the immunogenicity of proteins in humans or animals, comprising analysing the CD4+ T lymphocyte response specific to the protein to be tested in individuals of the (human or animal) species in which the immunogenicity of said cell is analysed.

The present invention relates to a method for evaluating the immunogenicity of proteins in humans or animals, which is based on analyzing the CD4+ T lymphocyte response specific for the protein to be tested in individuals of the (human or animal) species in which the immunogenicity of said protein is analyzed.

Proteins have the particularity of being potentially immunogenic, i.e. of being capable of triggering an immune response directed against themselves. According to the therapeutic activity expected, the immune response directed against a protein can be beneficial or, on the contrary, undesirable. It is obviously desired when the protein is a vaccine candidate, although it is dreaded when the protein has a therapeutic activity independent of the immune response.

Since 1995, proteins represent a third of the therapeutic products placed on the market. They have many advantages, in particular of specificity, for instance monoclonal antibodies which target receptors, or vaccines which induce immune responses specific for pathogenic agents. The immune responses induced by proteins have very varying consequences from one protein to another.

In some cases, the antibodies induced can reduce the efficacy of the protein (coagulation factor VIII, interferon-beta (IFN-beta)). In other cases, they can pose health problems, for instance erythropoietin or thrombopoietin for which the antibodies induced neutralize the endogenous molecule and cause effects opposite to those expected. Proteins can be allergenic and can cause allergies, the intensity of which varies from unpleasant local symptoms to systemic attacks of anaphylactic type which can put the patient's life in danger.

On the other hand, vaccine candidates can induce immune responses that are insufficient to be effective or in a limited number of patients.

Finally, the modern tools of protein engineering and of directed evolution, such as L-Shuffling make it possible to generate proteins having original sequences and increased biological activities. The immunogenicity of these new modified proteins is unknown.

The immunogenicity of a solution of a protein is not the result solely of the protein itself, but can also be the result of extrinsic factors (A. S. de Groot and D. W. Scott, TRENDS in Immunology, Oct. 27, 2007, Vol. 28). For example, the contaminating molecules present in protein preparations, for example the bacterial DNA (CpG) and the endotoxins contaminating preparations of recombinant proteins, and also protein aggregates, can promote an immune response against a protein preparation. In addition, protein modifications, such as modification of the amino acid sequence (mutations), denaturation, post-translational modifications (glycosylation, for example) and complexing or coupling with organic compounds (pegylation, for example), can, depending on their nature, either increase or decrease the immunogenicity of proteins.

These problems demonstrate the importance of predicting the immunogenicity of proteins before they are administered to humans or to animals and of evaluating the immunogenicity, preferably on the solution of the protein that will be injected.

In addition, nontherapeutic proteins (human proteins for humans or proteins of the same specifies for animals) can become immunogenic following a modification of their structure by organic compounds, in particular therapeutic compounds. This is because complexing or coupling of proteins (human serum albumin, for example) with certain therapeutic molecules (hapten) or with haptenic metabolites (penicilloyl group of antibiotics of the beta-lactam family) can occur in vivo after administration of organic molecules and can trigger, in humans, immune reactions that can range up to allergic responses (U. Kragh-Hansen, Pharmacol. Rev., 1981, 33, 17-53; H. Bundgaard and J. Hansen, J. Pharm. Pharmacol., 1982, 34, 304-309; Lafaye and C. Lapresle, J. Clin. Invest., 1988, 82, 7-12; Brander et al., The Journal of Immunology, 1995, 155, 2670-2678; P; J. P. Thyssen and H. I. Maibach, Contact Dermatitis, 2008, 59, 195-202). These responses are T-lymphocyte-dependent and compound-specific. In fact, the T cells recognize them in the form of modified peptides. These peptides are either directly modified at the surface of the antigen-presenting cell on the HLA molecule, or result from the degradation of modified proteins.

Consequently, it is also important to predict the immunogenicity of proteins modified by therapeutic compounds before these compounds are administered to humans or to animals.

Immune responses against proteins involve three different cell types which, by cooperating, result in the production of protein-specific antibodies.

B lymphocytes are the cells which produce antibodies. Each B lymphocyte produces a single antibody molecule. The sequence of this antibody results from the immunoglobulin gene recombination which occurs in each B lymphocyte precursor. Given the high number of immunoglobulin genes and the combination of rearrangements, a large antibody repertoire is thus produced and makes it possible to recognize the majority of molecules presented to the immune system. Naive B lymphocytes (i.e. lymphocytes which have never seen the antigen) have a surface IgM which allows them to recognize a protein. During the first contact with the protein, recognition of said protein causes the primary response characterized by the secretion of IgM specific for the protein. The secondary response occurs during the subsequent contacts. It is characterized by the secretion of other classes of immunoglobulins, in particular IgGs and IgEs specific for the protein, and requires the involvement of CD4+ T lymphocytes which are also specific for the protein.

Dendritic cells (DCs) are professional antigen-presenting cells (APCs) which were discovered by Steinman in 1973 (J. Exp. Med., 1973, 137:1142-1162). DCs exist in two forms: immature DCs and mature DCs. Immature DCs have a high capacity for endocytosis and play the role of sentinelles in the peripheral tissues. Mature DCs are involved in the initiation of immune responses owing to the presentation by the molecules of the major histocompatibility complex (MHC), to the T lymphocytes, of the peptides derived from the antigens that they have endocytosed in the immature state. Dendritic cells express the DC-SIGN marker and also MHC molecules (HLA molecules in humans). It is possible to distinguish mature DCs from immature DCs by surface markers. Mature DCs in fact express costimulatory molecules such as CD83, CD86 and CD40, whereas said molecules are expressed to a lesser extent on immature DCs. The peptide presentation function is provided by the MHC class I molecules (HLA I in humans) with respect to CD8+ T lymphocytes and MHC class II molecules (HLA II in humans) with respect to CD4+ T lymphocytes. There are four types of HLA class II molecules: two HLA-DR types, one HLA-DQ type and one HLA-DP type. These molecules exhibit strong sequence homologies and adopt a common structure. However, they are highly polymorphic since each type of HLA class II molecule has numerous alleles throughout the world. In 2008, there were 640 DRB alleles, 91 DQB1 alleles and 128 DPB1 alleles. This diversity has a direct effect on the function of peptide presentation by HLA class II molecules since the sequence variations are mainly located in the peptide-binding site. Owing to these sequence variations, each molecule binds a peptide repertoire which is specific thereto. This is why the localization of the CD4+ T epitopes, which are the peptides recognized by CD4+ T lymphocytes, depends on the HLA class II molecules of each individual and varies from one individual to another.

CD4+ T lymphocytes are the principal cells which control the appearance of antibodies. These cells are characterized by the expression of CD3, which is specific to T lymphocytes, and CD4, which distinguishes them from CD8 T lymphocytes. They are protein-specific cells. They are in fact specifically activated by the peptides derived from the proteins that the HLA class II molecules present to them. Recognition of the HLA class II molecule/peptide complexes occurs via the T receptor. The T receptor is a rearranged receptor which has a structure close to that of immunoglobulins. It is generated by a rearrangement of the T receptor genes, the T receptor being specific to each T lymphocyte. The activation of CD4+ T lymphocytes is reflected by the secretion of cytokines, such as IL-2, IL-4 or IFN-γ, and the appearance of costimulatory molecules such as CD40L.

Cell cooperation between these three cell types results in the production of antibodies directed against the proteins.

The protein is taken up by the immature dendritic cells and is degraded to peptides. Among the peptides generated, some have anchoring residues suitable for binding to MHC class II molecules and for being presented to CD4+ T lymphocytes. Under the effect of inflammatory factors that can result from administration of the protein, the immature dendritic cells differentiate into mature dendritic cells and become capable of stimulating naive CD4+ T lymphocytes. They migrate to the lymph nodes, where they encounter the CD4+ T lymphocytes. CD4+ T lymphocytes which have a T receptor specific for the peptide/MHC class II molecule complexes are activated by the presentation of the peptides derived from the protein and presented by the MHC class II molecules at the surface of the mature dendritic cells. They then change from a naive CD4+ T lymphocyte state to that of experienced CD4+ T lymphocytes or activated CD4+ T lymphocytes. The protein also reaches the B lymphocytes. The B lymphocytes which have a surface immunoglobulin suitable for recognition of the protein internalize the protein, degrade it and generate peptides. According to a process similar to that described in dendritic cells, peptides bind to the MHC class II molecules and are presented to the specific and experienced CD4+ T lymphocytes. The recognition of these peptides activates them and causes them to provide, by means of cell contacts (CD40/CD40L) and by means of the cytokines that they secrete, the necessary assistance to the differentiation of B lymphocytes into plasma cells which secrete antibodies specific to the protein. These antibodies produced against the protein can neutralize its activity and modify its pharmacokinetics.

CD4+ T lymphocytes therefore contribute in a major way to the triggering of the humoral response against proteins. It is in fact their recruitment which enables the secretion of protein-specific antibodies by B lymphocytes. Several observations made in hemophiliacs illustrate the major role of CD4+ T lymphocytes in the appearance of neutralizing antibodies. Hemophilia A is due to a deficiency in coagulation factor VIII and is treated with regular injections of factor VIII. More than 30% of hemophiliacs with severe hemophilia develop a humoral response which inhibits the action of factor VIII. The inhibitory antibodies are IgGs (Jacquemin et al., Blood, 1998, 92:496-506), the appearance of which is known to be dependent on CD4+ T lymphocytes. Indeed, mutations are observed in the genes encoding the variable parts of anti-factor VIII antibodies (Jacquemin et al, Blood, 1998, 92:496-506). The appearance of these mutations is a CD4+ T lymphocyte-dependent phenomenon. In hemophiliac individuals with a defined anti-factor VIII antibody titer, infection with the AIDS virus causes the disappearance of the specific antibodies when the CD4+ T lymphocyte titer decreases (Bray et al., Am. J. Hematol., 1993, 42:375-379). Owing to the major role of specific CD4+ T lymphocytes, the ability of a protein to stimulate naive CD4+ T lymphocytes is expected to reflect its ability to induce antibodies. Thus, studies carried out in animals have shown that peptides (Herve et al., Mol. Immunol., 1997, 34:157-163) and proteins (Yeung et al., J. Immunol., 2004, 172:6658-6665) which, after modification of their sequences, have lost the ability to induce the activation of naive CD4+ T lymphocytes, do not induce antibodies (Herve et al., Mol. Immunol., 1997, 34:157-163; Yeung et al., J. Immunol., 2004, 172:6658-6665).

This activation is the result of at least three different phases which each have an influence on the activation of CD4+ T lymphocytes (Adorini et al., J. Exp. Med., 1988, 168:2091-2104):
1. processing of the protein, which comprises its uptake by immature dendritic cells and its degradation to peptides,
2. binding of the peptides to the MHC class II molecules (HLA II in humans), and
3. recognition, by naive CD4+ T lymphocytes, of the peptides presented by the MHC class II molecules (HLA II in humans) at the surface of mature dendritic cells.

Consequently, methods for evaluating the immunogenicity of proteins seek to reproduce the activation of human naive CD4+ T lymphocytes.

### Animal Models

The animal models are mainly the mouse, the rat, the rabbit or the monkey. In order to evaluate the immunogenicity of proteins, the proteins are injected into animals several times and according to various routes of administration, and the appearance of antibodies is investigated. The prediction for the immunogenicity of therapeutic proteins is considered to be low. This is because, since the antibody response is dependent on CD4+ T lymphocytes, the differences in specificity between the MHC molecules of animals and the HLA class II molecules of humans leads to important differences in immunogenicity. The nature and the number of peptides presented by the MHC class II molecules of animals and recognized by the CD4+ T lymphocytes are different than those presented by the HLA class II molecules. In addition, the B-lymphocyte and T-lymphocyte repertoire of animals is formed by selection processes which can result in a repertoire different than that of humans. T and B lymphocytes undergoing differentiation are in fact eliminated from the bloodstream by the presentation of self molecules. These even subtle differences in composition of the repertoire cause considerable differences in response, in particular with respect to human proteins.

### In Silico Methods

In silico methods identify peptides capable of binding to HLA class II molecules and therefore attempt to reproduce only the second phase of the process (Sturniolo et al., Nat. Biotechnol., 1999, 17:555-561; Koren et al., Clin. Immunol., 2007, 124:26-32). These methods are based on the presence of motifs specific to HLA class II molecules or on prediction scores established on the basis of matrices reflecting the influence of the 20 amino acids at each position on binding or on expert systems. Other approaches are based on a structural analysis of the HLA molecule/peptide complexes (Desmet et al., Proteins, 2005, 58:53-69). Each of these approaches generates both false positives and false negatives with respect to the binding of the peptides to HLA class II molecules. A recent comparative study shows that the predictive value of these tests for identifying peptides having an ability to stimulate CD4+ T lymphocytes is low, which means that the number of false positives is high and not very satisfactory (Wang et al., PLoS. Comput. Biol., 2008, 4:e1000048).

### Biochemical Methods

In vitro biochemical methods identify peptides on the basis of their affinity for HLA class II molecules. Like the in silico methods, these approaches suffer from relating only to the second phase of the CD4+ T lymphocyte activation process. Peptides which bind to HLA class II molecules may not induce CD4+ T lymphocyte activation (Castelli et al., Eur. J. Immunol., 2007, 37:1513-1523), such that this method also suffers from a high number of false positives.

### Cellular Methods

It is also possible to use in vitro cell assays for evaluating the immunogenicity of proteins in humans. These approaches are based on the specific activation of CD4+ T lymphocytes in individuals who have never been in contact with the antigen (Tangri et al., J. Immunol., 2005, 174, 3187-3196; Stickler et al., Environ. Health Perspect., 2003, 111:251-254; Jaber, A. and M. Baker, J. Pharm. Biomed. Anal., 2007, 43:1256-1261; patents EP 1512004, EP 1483581, EP 1073754).

Human presenting cells are incubated in the presence of antigens (peptides or proteins) and then cultured with CD4+ lymphocytes derived from the same donor. The CD4+ T lymphocytes specific for the antigens are stimulated and proliferate. Their number increases such that they become detectable by means of a cell assay, such as tritiated thymidine incorporation or cytokine secretion. These approaches therefore attempt to reproduce, in vitro, the activation and amplification of the protein-specific CD4+ T lymphocytes that could take place in vivo in the individual to whom the protein is intended to be administered.

### Cellular Methods Using Only Peptides

Several methods for evaluating the immunogenicity of proteins and for engineering proteins which have a low immunogenicity are based on the use of peptides for stimulating naive CD4+ T lymphocytes and detecting the protein-specific CD4+ T lymphocytes thus activated (Stickler et al., Environ. Health Perspect., 2003, 111:251-254; patents EP 1512004, EP 1483581, EP 1073754). The peptides used cover the entire sequence of the protein studied. The advantages of using peptides are the fact that they have very good reactivity in activation assays, they are easy to produce and store, and they offer the possibility of localizing the immunogenic sequences. However, they have many drawbacks in terms of evaluating the immunogenicity of a protein. In order to cover the sequence of the protein as well as possible, a large number of peptides must be synthesized and tested, which inevitably increases the cost of the immunogenicity evaluation tests. In addition, since the optimum size of the peptides for activating CD4+ T lymphocytes is very variable from one CD4+ T epitope to another, it is possible that the collection of peptides covering the sequence of the protein does not contain the optimum peptides and does not therefore make it possible to detect all the CD4+ T lymphocytes specific for the protein. Conversely, the use of peptides during CD4+ T lymphocyte activation phases risks activating CD4+ T lymphocytes which are not specific for the protein and can therefore generate false-positive responses. This is because these approaches do not take into account the step of uptake and degradation of the peptides in the presenting cell. The peptides do not require processing by the presenting cell and are loaded onto the HLA molecules present at the surface of the presenting cell. Although they are contained in the sequence of the protein, epitopes may in fact not be specific for the protein. Such epitopes are called cryptic epitopes (Gammon et al., Immunol. Rev., 1987, 98:53-73). They induce CD4+ T lymphocytes which are specific for themselves, but these CD4+ T lymphocytes cannot recognize the protein presented by the presenting cells. These epitopes may correspond to peptides which are degraded during the processing by the presenting cell (Adorini et al., J. Exp. Med., 1988, 168:2091-2104) or to peptides of which the equivalent sequence in the protein bears a post-translational modification which prevents its presentation. The use of peptides during CD4+ T lymphocyte activation phases therefore risks overestimating the immunogenicity of the proteins.

In addition, a protein is derived from a defined production process. It may undergo post-translational modifications such as glycosylation. The nature and the extent of the glycosylation depend on the host cell used to produce the protein. During its production and its storage, a protein can undergo modifications such as methionine and tryptophan oxidation. Finally, it can contain contaminating molecules which participate in the immunogenicity of the preparation.

For these reasons, it appears to be essential to carry out the immunogenicity tests on the preparation which contains the protein and not on synthetic peptides.

### Cellular Methods Using Proteins and Peptides

Tangri et al. describe a method for evaluating the immunogenicity of proteins in humans using peripheral blood mononuclear cells (PBMCs) from naive donors, comprising:
Step 1: separation of the monocytes and of the CD4+ T lymphocytes,
Step 2: preparation of dendritic cells (DCs) loaded with the protein by culturing the monocytes for 7 days in the presence of GM-CSF and of IL-4, loading the DCs with the protein for several hours, and then washing and centrifuging the DCs. Monocytes cultured under the same conditions, with the exception of the loading step, which is carried out in a medium not containing the protein, are used as a control,
Step 3: stimulation of the CD4+ T lymphocytes by coculture with DCs loaded with the protein (initial stimulation), and then with PBMCs loaded with the protein or with immunogenic peptides derived from said protein (two restimulations),
Step 4: detection of the CD4+ T lymphocyte response specific to said protein by means of an ELISPOT-IFN-γ assay which comprises the coculture of the stimulated CD4+ T lymphocytes with PBMCs loaded with peptides derived from said protein, in plates coated with anti-IFN-γ antibodies, and then the immunoenzymatic detection of the IFN-γ-producing cells.

The epitopes recognized by the CD4+ T lymphocytes are identified using overlapping peptides covering the entire sequence of the protein to be studied and by means of naive donors of varied HLA II haplotype. The frequency of responding individuals is determined for each peptide.

This method is sensitive and specific given that it makes it is possible to compare the immunogenicity of a wild-type protein with that of variants of which the immunogenicity has been reduced by modification of the affinity for HLA-DR molecules of immunodominant epitopes of this protein. However, since step 4 of demonstrating the specificity of the CD4+ T lymphocytes is carried out by means of peptides, it does not make it possible to detect the possible presence of contaminating molecules which participate in the immunogenicity of protein preparations and to take into account the formulation of the protein, or the existence of post-translational modifications, of possible modifications of amino acids (such as oxidation) or of modifications introduced into the protein, such as pegylation of the galenic form of the protein.

In addition, for comparing the various variants, it has the major drawback of using antigenic peptides corresponding to CD4+ T epitopes of the test protein, which implies prior identification of these CD4+ T epitopes using peptides covering the entire sequence of the protein to be studied, which is long, laborious and expensive.

### Cellular Methods Using Only Proteins

Such a method for evaluating immunogenicity has recently been described by Jaber and Baker (Jaber, A. and M. Baker, J. Pharm. Biomed. Anal., 2007) and derives from prior studies described by others (Schlienger et al., Blood, 2000, 96:3490-3498).

This method is simpler and faster than the method described by Tangri et al. In addition, it does not use peptides, but only the protein to be analyzed. More specifically, the method described by Jaber, A. and M. Baker comprises step 1 as described above, with step 2 comprising an additional step of incubation of the protein-loaded immature DCs for 24 h in the presence of a maturing agent (TNF-α) so as to obtain mature DCs loaded with the protein. Step 3 of stimulating the CD4+ T lymphocytes by coculture with mature DCs loaded with the protein to be analyzed does not comprise any restimulation. Step 4 for detecting the CD4+ T lymphocyte response specific to said protein is carried out concomitantly with step 3 of stimulating the CD4+ T lymphocytes (at the end of step 3, i.e. after 6 to 7 days of coculture) by means of an ELISPOT-IFN-γ assay or a lymphocyte proliferation assay.

The protein-loading of the DCs is carried out before the induction of their maturation since it has been shown that loading immature DCs with the antigen before inducing their maturation is essential for obtaining mature DCs capable of efficiently activating naive CD4+ T lymphocytes (Schlienger et al., Blood, 2000, 96, 3490-3498).

This method has several drawbacks. Specifically, the authors use, as a positive control, a protein known to be highly immunogenic (KLH: Keyhole Limpet Hemocyanin) and which serves in particular as a reference in vaccination trials. The response obtained against the KLH is grater than that of the background noise (CD4+ T lymphocyte response in the presence of control DCs) only by a factor of 1.4 in the ELISPOT assay and by a factor of 2.1 in the proliferation assay. These signal-to-background-noise ratios are low and indicate that the method is not very sensitive. A protein less immunogenic than KLH, as is the case for the beta-1a interferons tested in this study, in fact gives rise to a response that is not very different than the background noise. It can also be noted that the response of one of the proteins (RNF2) is even lower than the background noise, which emphasizes the low specificity of this method.

It therefore appears that the non-cellular methods and the cellular methods using only peptides suffer from major drawbacks which do not make it possible to obtain reliable results.

The cellular methods which use proteins combine all the phases of CD4+ T lymphocyte activation and reproduce the entire process of activation of human naive CD4+ T lymphocytes. However, only the mixed method which uses proteins and peptides is sensitive and specific. The cellular method which uses only proteins is not very specific and not very sensitive.

Consequently, the cellular methods for evaluating the immunogenicity of proteins that are currently proposed do not make it possible to obtain sensitive and specific results without being free of the use of peptides, which is expensive and may conceal the effects due to accidental or intentional modifications of the natural sequences of therapeutic proteins. In addition, they do not make it possible to detect the possible presence of contaminating molecules which participate in the immunogenicity of protein preparations, and they do not take into account the formulation of the protein or the method for producing said protein.

The objective of the present invention is to overcome the drawbacks of the prior art methods by providing a specific and sensitive method for evaluating the immunogenicity of proteins in vitro, which uses the test protein as sole antigen for measuring the CD4+ T lymphocyte response specific for this protein.

The inventors have demonstrated that measuring the CD4+ T lymphocyte response specific for a protein using naive CD4+ T lymphocytes requires prior activation of the CD4+ T lymphocytes in a step separate from the detection of the CD4+ T lymphocyte response specific for this protein. In addition, the choice of the presenting cells for the test antigen (protein) and also the preparation of said cells makes it possible to improve the sensitivity and the specificity of this specific CD4+ T lymphocyte response.

The method of the invention measures the immunogenicity of a protein preparation by taking into account the specificity, for said protein preparation, of the CD4+ T lymphocytes activated by said preparation. This direct measurement of the immunogenicity of proteins observes the physiological processing of the protein in the presenting cell and is more effective.

The sensitivity and the specificity of the method of the invention are demonstrated in Table I. High response strengths are observed with proteins known to be immunogenic in humans (OVA, KLH and murine monoclonal antibodies Ma-2,3 (Trémeau et al., FEBS Lett., 1986, 208 (2), 236-240)). On the other hand, the response strengths are very low or zero with proteins that are nonimmunogenic in humans (human insulin, human antitrypsin, human antithrombin). The method according to the invention has been used successfully for comparing the immunogenicity of various human or chimeric (human-murine) therapeutic proteins and of proteins known to be immunogenic in humans (KLH and a murine monoclonal antibody, Mα2,3). Thus, the method according to the invention has made it possible to classify the relative immunogenicity of a series of therapeutic proteins, compared with that of KLH and of the murine antibody Mα2,3. As shown in Tables II and V, the values of relative immunogenicity of the various proteins, obtained according to the method of the invention, are correlated with the values obtained clinically (percentage of individuals having specific antibodies).

The method according to the invention makes it possible to evaluate the immunogenicity of proteins and in particular to classify the immunogenicity of a first protein relative to the immunogenicity of at least a second protein. The invention is notable in that it makes it possible to screen for candidate proteins and to compare their immunogenicity relative to a reference before they are administered to humans. The method according to the invention makes it possible in particular to select a protein which has been improved by directed evolution while at the same time having an immunogenicity which is comparable to or lower than the reference protein.

The method according to the invention can be used to screen for therapeutic proteins having an immunogenicity which is reduced, neutralized or higher compared with the reference protein that is very immunogenic, for instance KLH, or not very immunogenic, such as human insulin.

The method according to the invention can be used, conversely, to screen for vaccine proteins which induce immune responses that are sufficiently effective compared with the reference vaccine protein.

The method according to the invention can also be used for comparing the immunogenicity of a protein according to the method for the production thereof, the formulation thereof and the production batch thereof.

The method according to the invention can also be used for comparing the immunogenicity of a protein which has been modified with respect to a reference (unmodified) protein and thus evaluating the effect of the modifications introduced into a protein on the immunogenicity of this protein.

A subject of the present invention is a method for evaluating the immunogenicity of proteins, comprising at least the following steps:

a) activating CD4+ T lymphocytes of at least one donor by coculturing CD4+ T lymphocytes of each donor with autologous antigen-presenting cells loaded with a test protein, in at least ten independent culture vessels (n≧10) for each donor,
b) measuring the activation of said CD4+ T lymphocytes with respect to autologous immature dendritic cells loaded with said protein (value Ai with 1≦i≦n) and measuring, in parallel, the activation of said CD4+ T lymphocytes with respect to non-loaded autologous immature dendritic cells (value Bi with 1≦i≦n), for each of the n cultures of CD4+ T lymphocytes of each donor of step a), and
c) calculating the value of the immunogenicity of said protein by comparison of all the values Ai with all the values Bi, obtained for all the cultures of CD4+ T lymphocytes of the donors of step a) (n cultures per donor).

### Definitions

The term “antigen” is intended to mean any molecule, in particular a protein or a peptide, which can be recognized by the immune system, and in particular by the CD4+ T lymphocytes.
The term “antigen-presenting cell or APC” is intended to mean a cell expressing one or more MHC class II molecules (HLA class II molecules in humans) and capable of presenting the antigens to CD4+ T lymphocytes specific for this antigen. As antigen-presenting cells, mention may in particular be made of dendritic cells (DCs), peripheral blood mononuclear cells (PBMCs), monocytes, macrophages, B lymphocytes, lymphoblastoid lines, and genetically modified human or animal cell lines expressing MHC class II molecules, in particular HLA II molecules.
The term “protein” is intended to mean any natural, synthetic or recombinant protein. This term also encompasses modified proteins resulting in particular from the modification of the amino acid sequence (mutations, for example) of a protein, from the introduction of post-translational modifications (glycosylation, for example) into a protein, from the denaturation of a protein (disorganization of the spatial structure without breaking of the covalent bonds) and from the complexing or coupling (formation of a conjugate) of a protein with an organic compound such as for example, a therapeutic molecule or a metabolite derived from this molecule.
The expression “antigen-presenting cell loaded with a protein or loaded with protein” is intended to mean a presenting cell which has internalized said protein (endocytosis) and degraded it to peptides (proteolytic degradation) which have been loaded onto MHC class II molecules and are presented at the surface of said presenting cell in the form of MHC II/peptide complexes.
The expression “loading a presenting cell with protein” is intended to mean the coincubation of said presenting cell with a protein under conditions which allow the internalization, degradation, loading onto MHC II molecules and presentation of said protein in the form of complexes of MHC II/peptide derived from said protein, expressed at the surface of said presenting cell. The non-loaded presenting cells are prepared in parallel, in the absence of the protein.
The term “CD4+ T lymphocyte” is intended to mean a CD4+ T lymphocyte which may or may not have been in contact with the antigen.
The term “naive CD4+ T lymphocyte” is intended to mean a T lymphocyte which has never been in contact with the antigen.
The term “dendritic cell or DC” is intended to mean an antigen-presenting cell capable, by presenting the antigen, of stimulating CD4+ T lymphocytes specific to this antigen.
The term “immature dendritic cell or iDC” is intended to mean a dendritic cell capable of internalizing and degrading proteins.
The term “mature dendritic cell or mDC” is intended to mean a cell resulting from the maturation of an iDC by a maturation agent and capable, by presenting the antigen, of stimulating CD4+ T lymphocytes specific to this antigen even if these CD4+ lymphocytes are naive. The mDCs are characterized by the expression “at their surface” of costimulatory molecules such as CD83, CD86 and CD40 and of a high amount of MHC II/peptide complexes (CD40+, CD83^{hi}, CD86^{hi}, MHC class II^{hi}).
The term “maturation agent” is intended to mean a molecule of a mixture of molecules capable of inducing the maturation of immature DCs into mature DCs.
The term “activation, stimulation or induction of CD4+ T lymphocytes” is intended to mean the stimulation of CD4+ T lymphocytes having a T receptor specific for peptide/MHC class II molecule complexes presented at the surface of antigen-presenting cells. This activation is reflected by proliferation of the CD4+ T lymphocytes, or the secretion of cytokines such as IL-2, IL-4 or IFN-γ, or the appearance of costimulatory molecules such as CD40L.

When the CD4+ T lymphocytes are naive, the activation is optimal when the antigen-presenting cells are mature dendritic cells presenting peptide/MHC class II molecule complexes recognized by the T receptor of said CD4+ T lymphocytes.
The term “T lymphocyte line” is intended to mean all the T lymphocytes contained in an independent culture vessel, in particular in a culture plate well.
The term “T lymphocyte line specific for a protein” is intended to mean a T lymphocyte line which is activated significantly by DCs loaded with the protein, compared with the same DCs which are not loaded.
The term “donor” or “CD4+ T lymphocyte donor” is intended to mean a human or animal individual, in particular a human or non-human mammal, from which are derived the CD4+ T lymphocytes which are cultured in step a) of the method of the invention.
The term “autologous cells” is intended to mean cells derived from the same human or animal individual, in particular a human or non-human mammal.
The term “immunogenicity” of a protein is intended to mean its ability to induce a specific immune response, in particular the induction of the activation of CD4+ T lymphocytes specific for said protein.
The term “evaluating the immunogenicity” is intended to mean measuring the CD4+ T lymphocyte response specific for the protein tested. This value is determined by its strength and by the frequency of responder individuals.
The strength of the specific CD4+ T lymphocyte response is calculated on the basis of the difference or the ratio between the values Ai and the values Bi, according to one of the following methods:
   (1) arithmetic mean of the differences between the values Ai and Bi (mean of the (Ai−Bi)) or (arithmetic mean of the Ais)−(arithmetic mean of the Bis):

$$\frac{( A 1 - B 1 ) + ( A 2 - B 2 ) + … + ( An - Bn )}{n}$$

$$or$$

$$\frac{( A 1 + A 2 + … + An ) - ( B 1 + B 2 + … + Bn )}{n}$$

(2) arithmetic mean of the quotients Ai over Bi:

$$\frac{( A 1 / B 1 + A 2 / B 2 + … + An / Bn )}{n}$$

(3) quotient of the arithmetic mean of the Ais over the arithmetic mean of the Bis:

$$\frac{( A 1 + A 2 + … + An )}{( B 1 + B 2 + … + Bn )}$$

(4) percentage of positive culture vessels, i.e. those for which the value A is at least double the value B (Ai/Bi≧2) with Ai≧S where S measures the background noise of the technique used for measuring the activation of the CD4+ T lymphocytes (step b). S varies according to the specificity of this technique. For example, when the activation is measured by ELISPOT, S corresponds to 15 spots per 10 000 CD4+ T lymphocytes.
(5) frequency of CD4+ T lymphocytes specific to the test protein among the CD4+ T lymphocytes of the donor. The distribution of the specific CD4+ T lymphocytes follows a Poisson law with parameter λ, where λ is the average number of specific CD4+ T lymphocytes per well. The probability P(X=k) of there being k specific CD4+ T lymphocytes in a well is given by the formula: P(X=k)=e^{-λ}λ^{k}/k!. It is then possible to calculate the probability that a well does not contain specific cells by taking the value k=0, this value is P(X=0)=e^{-λ}. The value of λ=ln(P(X=0)), which depends on the number of negative wells, is deduced therefrom. The number of negative wells is in fact an estimation of P(X=0). This number of negative wells can be easily calculated. The number of specific CD4+ T lymphocytes per number of CD4+ T lymphocytes distributed is then obtained from the number of CD4+ T lymphocytes distributed per well and from the value calculated for λ. The average frequency is in particular expressed as number of specific CD4+ T lymphocytes per million CD4+ T lymphocytes.

When the immunogenicity of a protein is measured in parallel on several individuals, the immunogenicity of this protein for the collection of individuals tested can be evaluated directly by performing the abovementioned calculations ((1), (2), (3) or (4)) on all of the wells analyzed for all the individuals of the collection (sum of the values n of all the individuals). Alternatively, the above-mentioned calculations can be performed for each individual, and then the arithmetic mean of the values obtained is subsequently calculated.

The strength of the CD4+ T response is significant (specific response) when the mean of the differences (Ai−Bi) is greater than or equal to S, the mean of the quotients Ai/Bi or the quotient of the mean of the Ais over the mean of the Bis is greater than or equal to 2 or at least one of the culture vessels tested is positive.
The frequency of responder individuals. This frequency corresponds to the percentage of individuals of a collection of individuals tested for whom the strength of the CD4+ T response is significant.
The term “culture vessel” is intended to mean any support suitable for cell culture, in particular culture vessels comprising independent culture chambers, such as, in particular, culture plates containing independent culture wells. These are, for example, 6-, 24- or 96-well culture plates.

In accordance with the method of the invention, the CD4+ T lymphocytes and the autologous antigen-presenting cells are derived from one or more individuals (donor(s)) of the species in which the immunogenicity of the protein is analyzed. Preferably, they are human individuals or non-human mammalian individuals, such as, in a nonlimiting manner, laboratory animals (mice, rats, rabbits, monkeys), domestic animals (dogs, cats, guinea pigs, horses, cows, pigs, sheep, goats) or wild animals (felines, pachyderms, hoofed animals).

Generally, the donor is a healthy individual that has not been in contact with the protein (individual naive with respect to said protein), but the method of the invention may also be applied to healthy donors that have been in contact with the protein (individuals vaccinated with the protein) or to patients that have not been in contact with the protein or that have been in contact with the protein (therapeutic protein, allergen, tumor allergen, autoantigen).

According to one advantageous embodiment of said method, the CD4+ T lymphocytes and the autologous antigen-presenting cells are derived from an individual who is naive with respect to said protein. Preferably, it is a human individual who is naive with respect to said protein.

The test protein is any protein capable of inducing a specific immune response in the donor, such as a modified or unmodified protein as defined above. It is in particular a protein which is intended to be administered to the donor (vaccine antigen, protein having a therapeutic activity which targets the immune system or protein having a therapeutic activity independent of the immune system) or is capable of being in contact with the donor (allergen, autoantigen).

According to another advantageous embodiment of said method, the test protein is a therapeutic protein. It is in particular a vaccine antigen which induces an immune response specific for a pathogenic agent or for a tumor or for a protein having a therapeutic activity which targets the immune system or which is independent of the immune system (human, chimeric or humanized monclonal antibody, cytokine, etc.).

The test protein may be isolated or included in a mixture, in particular in a mixture comprising different proteins.

The CD4+ T lymphocytes and the autologous antigen-presenting cells are cultured under standard conditions (temperature, CO₂), in a suitable conventional culture medium. The CD4+ T lymphocytes and the antigen-presenting cells are in particular isolated from the peripheral blood mononuclear cell (PBMC) fraction of at least one individual of the species in which the immunogenicity of the protein is evaluated, according to conventional techniques known to those skilled in the art, as described, in particular, in Castelli et al., European Journal of Immunology, 2007, 37, 1513-1523. The PBMCs are isolated by density gradient centrifugation (Ficoll gradient) and then cultured so as to separate the adherent cells (monocytes) and the non-adherent cells (lymphocytes). The isolated cells (PBMCs, monocytes, lymphocytes, CD4+ T lymphocytes, dendritic cells) can be cultured immediately, or frozen in a suitable standard medium and cultured subsequently.

The CD4+ T lymphocytes are purified from the non-adherent mononuclear cells by positive selection using anti-CD4 antibodies, in particular using anti-CD4 antibodies coupled to magnetic beads.

The adherent cells (monocytes) are used to prepare (immature and mature) dendritic cells. Typically, the immature dendritic cells are obtained by culturing mononuclear cells that adhere to the plastic, for 3 to 7 days, preferably 5 days, in the presence of IL-4 (1000 IU/mL) and of GM-CSF (1000 IU/ml). The immature dendritic cells are then differentiated into mature dendritic cells by culturing in the presence of a maturation agent, for example LPS (E. coli lipopolysaccharide), TNF-α, CD40L or prostaglandin E2, for a period which varies according to the concentration of maturation agent used and can be readily determined for each maturation agent (TNF-α: 20 ng/ml for 24 h; LPS: 1 μg/ml for 4 h to 48 h).

The loading of the antigen-presenting cells with protein comprises incubating the antigen-presenting cells with the protein (in general 10 to 500 μg/ml) in standard culture medium for antigen-presenting cells (monocytes, DCs or PBMCs), generally at 37° C. for a few hours. After loading, the presenting cells are generally washed before being put back in culture.

The loading of the dendritic cells with protein can be carried out before induction of their maturation or simultaneously.

When the loading of the dendritic cells with protein is carried out before induction of their maturation, the immature dendritic cells are coincubated with the protein for at least 4 h, and they are generally washed before being cultured in the presence of the maturation agent for the amount of time necessary for their maturation, which is defined according to the nature and the concentration of maturation agent, as specified above.

When the loading of the dendritic cells with protein is carried out simultaneously with the induction of their maturation, the immature dendritic cells are cultured with the protein and the maturation agent for the amount of time necessary for their maturation.

According to another advantageous embodiment of said method, the antigen-presenting cells loaded with test protein (step a)) are mature dendritic cells loaded with said protein.

According to a first advantageous arrangement of this embodiment, said mature dendritic cells have been obtained by maturation of immature dendritic cells in the presence of LPS.

According to a second advantageous arrangement of this embodiment, the mature dendritic cells loaded with the protein are obtained from immature dendritic cells which have been loaded with the protein and simultaneously matured with the maturation agent. The immature dendritic cells were simultaneously incubated with the protein and the maturation agent for at least 4 hours, preferably for 4 hours to 48 hours, preferably for 24 h.

As indicated above, the prior art methods load the DCs with protein before induction of their maturation, since it has been shown that loading immature DCs with the antigen before inducing their maturation is essential for obtaining mature DCs capable of activating naive CD4+ T lymphocytes effectively (Schlienger et al., Blood, 2000, 96, 3490-3498).

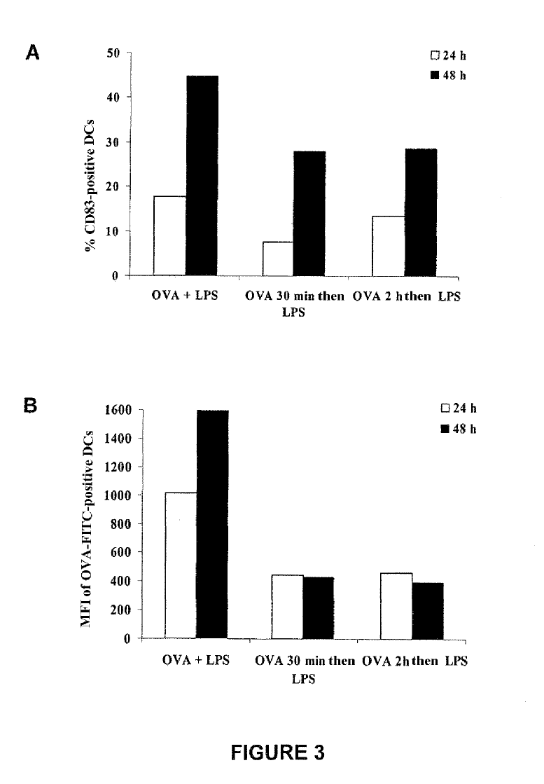

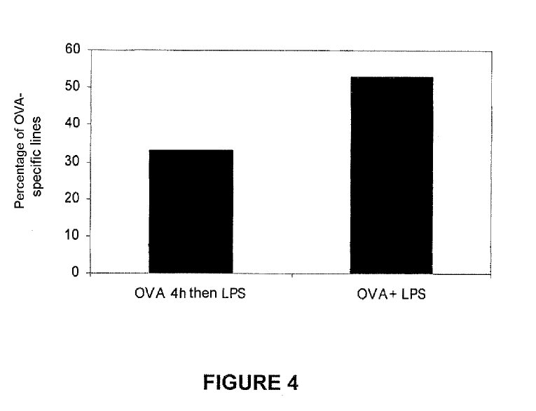

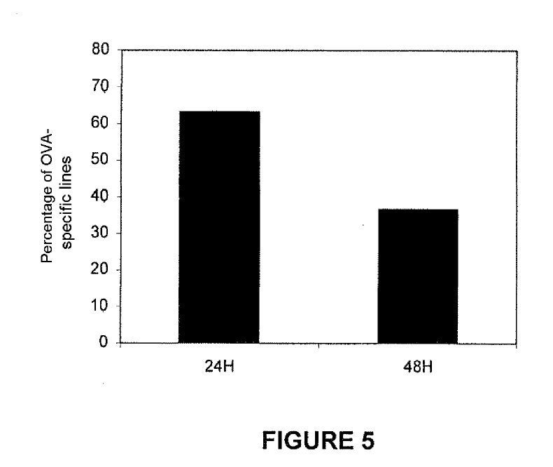

Surprisingly, the inventors have realized that loading of the immature DCs with the protein and simultaneous maturation of the immature DCs make it possible to effectively and rapidly activate the CD4+ T lymphocyte response while at the same time limiting the loss of cells and the risk of contamination of said cells. The inventors have shown, surprisingly, that the DCs can endocytose a protein continuously even if they have initiated their maturation, whereas incubation of the DCs with the protein before their maturation limits the uptake of the protein (FIG. 3B). In addition, simultaneously bringing the DCs and the protein into contact does not disturb their maturation, whereas the maturation is less efficient when the protein and the maturation agent are added separately (FIG. 3A). These steps of loading of the immature DCs with the protein and of simultaneous maturation make it possible to obtain, in an effective and optimum manner, DCs loaded with the protein of interest and rendered mature for the induction of specific CD4+ T lymphocytes. This method of effective activation of T lymphocytes by the proteins makes it possible to efficiently measure the specificity of T lymphocyte lines induced in the presence of proteins, which is used for evaluating the immunogenicity of the proteins (FIGS. 4 and 5).

In accordance with the method of the invention, each culture vessel of step a) comprises at least 10⁵ CD4+ T lymphocytes, preferably 10⁵ to 2×10⁵ CD4+ T lymphocytes. The number of antigen-presenting cells is less than that of the CD4+ T lymphocytes by at least a factor of 10, preferably by a factor of 10 to 20 (5×10³ to 2×10⁴ antigen-presenting cells). The cocultures are carried out in at least 10 independent culture vessels; preferably, they are carried out in 15 independent vessels, preferably they are carried out in independent vessels. The cocultures can in particular be carried out in the wells of a 96-well culture plate. The CD4+ T lymphocytes and the antigen-presenting cells are cocultured for at least five days.

According to another advantageous embodiment of said method, step a) comprises at least one restimulation of the CD4+ T lymphocytes by addition, to the coculture, of autologous antigen-presenting cells loaded with the test protein, as defined above. According to one advantageous arrangement of this embodiment, the restimulations are carried out every 5 to 7 days, after at least 5 days of coculture, preferably after 5 to 7 days of coculture. Preferably, step a) comprises two restimulations 5 to 7 days apart, the first being carried out after 5 to 7 days of coculture.

In accordance with the method of the invention, step b) comprises analyzing the specificity of the activated CD4+ T lymphocyte lines obtained in step a). Steps a) and b) are carried out separately. The activation of the CD4+ T lymphocytes is measured by culturing the activated CD4+ T lymphocytes obtained at the end of step a) with a new preparation of autologous antigen-presenting cells (immature DCs) loaded with the test protein.

The activation of the CD4+ T lymphocytes is measured using, as antigen-presenting cells, immature dendritic cells loaded with test protein, prepared as described above. Immature dendritic cells incubated in the absence of protein are used as a control for the specificity of the activated CD4+ T lymphocyte lines obtained in step a). The measurement of the activation of the CD4+ T lymphocytes comprises measuring the proliferation, the production of specific cytokine(s) (IL-2, IL-4 or IFN-γ) or the expression of CD4+ T lymphocyte activation markers (CD40L) by any conventional technique known to those skilled in the art. The proliferation of the CD4+ T lymphocytes can in particular be evaluated by measuring the incorporation of tritiated thymidine or the labeling with CFSE (5- and 6-carboxyfluorescein diacetate succimidyl ester). The production of specific cytokine(s) and the expression of activation markers can be evaluated by analysis of the transcripts (RT-PCR) or immunodetection of the corresponding protein in intracellular form or extracellular form (cytokines) or expressed at the membrane of the CD4+ T lymphocytes (activation markers), in particular by ELISA, RIA, ELISPOT or FACS, as described above (Castelli et al., European Journal of Immunology, 2007, 37, 1513-1523; Castelli et al., European Journal of Immunology, 2008, 38, 1-11).

According to another advantageous embodiment of said method, the activation of the CD4+ T lymphocytes (step b) is measured by means of a lymphocyte proliferation assay, an intracellular cytokine(s) labeling assay or an ELISPOT assay. According to one advantageous arrangement of this embodiment, step b) is carried out by means of an ELISPOT assay, preferably an ELISPOT-IFN-γ assay.

During the implementation of step b), the inventors have demonstrated various types of response profiles for the activated T lymphocytes (CD4+ T lymphocytes derived from step a)) and the importance of these differences in response in measuring the immunogenicity of a protein (step c).

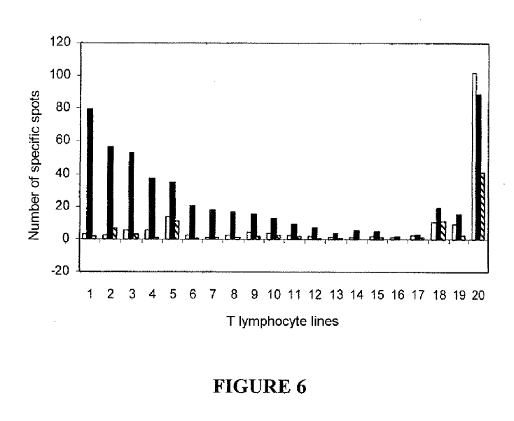

Specifically, the inventors have demonstrated (FIG. 6) that the activated CD4+ T lymphocyte lines derived from an individual exhibit three types of response profiles with respect to immature dendritic cells loaded with test protein and to non-loaded immature dendritic cells (without protein):

1) positive response with respect to the protein and negative response in the absence of protein; these are CD4+ T lymphocyte lines specific for the test protein (lines 1 to 9 of FIG. 6),
2) negative response in the presence or absence of the protein; these are CD4+ T lymphocyte lines not specific for the test protein (lines 10 to 17 of FIG. 6), and
3) positive response in the presence or absence of the protein; these are CD4+ T lymphocyte lines not specific for the test protein (lines 18 to 20 of FIG. 6).

The evaluation of the immunogenicity (step c)) takes into account the existence of these three response profiles. The immunogenicity can be expressed by the strength of the response and the frequency of responder individuals, determined as specified above. It makes it possible to distinguish at least four types of proteins: proteins having a high strength and a high responder frequency, proteins having a high strength but in a low number of individuals, proteins having a low strength with a high frequency of individuals and proteins having a low strength and a low responder frequency. Preferably, the value of the immunogenicity of the test protein compared with that of at least one standard protein of which the immunogenicity is known, serves to classify the test protein relative to a scale of immunogenicity values obtained for said standard proteins.

According to one particular arrangement of the above embodiments, said method comprises the following various steps:
step a):
obtaining immature dendritic cells (iDCs) and CD4+ T lymphocytes from a biological sample from the same donor,
obtaining mature DCs loaded with protein by loading of the iDCs with the test protein and simultaneous maturation of the iDCs with a maturation agent, preferably LPS, for at least 4 h, preferably 24 h,
coculturing said CD4+ T lymphocytes with said mature DCs loaded with protein, in at least 10 independent wells of a 96-well microplate, preferably 15 wells, for 5 to 7 days,
adding mature DCs loaded with protein, obtained as described above, to said coculture at least once, and preferably twice with a gap of 5 to 7 days;
step b): measuring the activation of said CD4+ T lymphocytes with respect to autologous immature dendritic cells loaded with said protein (value Ai with 1≦i≦n) and measuring, in parallel, the response of said CD4+ T lymphocytes with respect to non-loaded autologous immature dendritic cells (value Bi with 1≦i≦n), for each of the n cultures of CD4+ T lymphocytes of step a); preferably, the activation is measured by means of an ELISPOT-IFNγ assay;
step c): calculating the strength of the response, as specified above.

Preferably, the obtaining of iDCs and of CD4+ T lymphocytes in step a) comprises the following steps:
isolating mononuclear cells (PBMCs) from a peripheral blood sample from a donor, in particular by centrifugation on a Ficoll gradient,
culturing the mononuclear cells on a plastic support so as to isolate the adherent cells,
purifying the CD4+ T lymphocytes from the non-adherent cells, in particular by positive selection using anti-CD4+ antibodies coupled to a support such as magnetic beads, and
generating immature dendritic cells by culturing the adherent cells in the presence of GM-CSF and of IL4 for 3 to 7 days, preferably 5 days.

According to another advantageous embodiment of said method, steps a) to c) are carried out in parallel on cultures derived from a collection of different individuals, and step c) comprises calculating the strength of the response and the frequency of responders in the collection of individuals tested.

According to one advantageous arrangement of this embodiment, the collection of individuals tested is representative of a population of individuals in which the immunogenicity of the protein is analyzed. To do this, the collection of individuals is selected such that the frequencies of the HLA-DR alleles in the collection of individuals are close to those encountered in the population of individuals studied. The population of individuals corresponds, for example, to that to which the protein is intended to be administered. Alternatively, it is a population at risk, in particular and without this being exclusive, a population having a chronic pathological condition, a population for which a greater than normal proportion of immune response against the protein has been observed, or a population having antibodies against the protein.

According to another advantageous embodiment of said method, steps a) to c) are carried out successively or simultaneously with various test proteins and then the immunogenicity values obtained in step c) are compared with one another.

This embodiment makes it possible to compare the immunogenicity of several proteins. It makes it possible in particular to compare the immunogenicity of at least one test protein with that of a reference protein. It also makes it possible to compare the immunogenicity of similar proteins, in particular of variants of a protein, which have been improved by directed evolution, or of modified proteins as defined above. Thus, the method according to the invention makes it possible to screen for and select candidate proteins having the desired immunogenicity and to compare their immunogenicity relative to a reference before they are administered to humans. These proteins are in particular therapeutic proteins of which the activity is improved by directed evolution and which at the same time have an immunogenicity comparable to or even lower than the reference protein. There are also therapeutic proteins which have an immunogenicity that is reduced, neutralized or greater compared with the reference protein, which is highly immunogenic, for instance KLH, or not very immunogenic, such as human insulin. There are also vaccine proteins which induce immune responses that are sufficiently effective compared with the reference vaccine protein.

The method of the invention also makes it possible to compare the immunogenicity of the same protein at various steps of its production process, according to its production batch, according to its formulation or its method of production. Thus, the method according to the invention makes it possible to test the immunogenicity of a therapeutic protein during its production and its formulation.

The method of the invention also makes it possible to compare the immunogenicity of a therapeutic protein in various populations of individuals. This involves in particular comparing the immunogenicity of a therapeutic protein in a normal population and in a population at risk.

Thus, the method of the invention is of use for evaluating the immunogenicity of a therapeutic protein, whether during the development of a medicament: preclinical stage (selection of a candidate protein) and clinical stage (production, formulation), or throughout the life of this medicament, after marketing authorization for said medicament (specific studies on populations at risk or new target populations that are candidates for treatment/vaccination).

Thus, during the research and development stage, and in particular during the preclinical phases, the method allows immunological screening for future candidates predisposed to clinical studies. These future candidates may, for example, be selected by comparison of different variants of the same product with a reference protein which may be a protein of which the immunogenicity is known or a protein already used clinically. This method is also a valuable tool for evaluating the immunogenicity of bisimilars, in comparison with the reference product.

During clinical phases, this method makes it possible to define the immunogenic signature of a therapeutic product containing a therapeutic protein, that is to say, in other words, the intrinsic immunological characteristics of said product. This method thus enables a study on any sample of individuals that is representative of the population, in order to define in a significant manner the level of risk of immunogenicity of the product. It will then become possible to associate a risk of immunogenicity of said product with a particular HLA phenotype, if this risk exists, thus making it possible to identify populations at risk. Finally, the effects of any modifications of the therapeutic protein or of the method for preparing said protein on the immunogenicity of this therapeutic protein may also be monitored.

The change in the immunogenic signature of a therapeutic product may be evaluated after said product has been placed on the market. The term “change in the immunogenic signature” is intended to mean the study of the risk of immunogenicity of a product when the method for producing said product is modified, or when the use of said product is broadened to a pathological condition other than that for which it was designed.

The implementation of the method of the invention uses, unless otherwise indicated, conventional methods of immunology, cell culture, cell biology, molecular biology and recombinant DNA which are known to those skilled in the art. These techniques are described in detail in the literature; reference may be made, for example, to: Current Protocols in Molecular Biology (Frederick M. AUSUBEL, 2000, Wiley and Sons Inc, Library of Congress, USA); Current Protocols in Immunology (John E. Coligan et al., 2008, Wiley and Sons Inc, Library of Congress, USA), Molecular Cloning: A Laboratory Manual, Third Edition, (Sambrook et al, 2001, Cold Spring Harbor, N.Y.: Cold Spring Harbor Laboratory Press); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the series, Methods in ENZYMOLOGY (J. Abelson and M. Simon, eds.-in-chief, Academic Press, Inc., New York), specifically, Vols. 154 and 155 (Wu et al. eds.) and Vol. 185, “Gene Expression Technology” (D. Goeddel, ed.); Gene Transfer Vectors For Mammalian Cells (J. H. Miller and M. P. Calos eds., 1987, Cold Spring Harbor Laboratory); Immunochemical Methods In Cell and Molecular Biology (Mayer and Walker, eds., Academic Press, London, 1987); Handbook Of Experimental Immunology, Volumes I-IV (D. M. Weir and C. C. Blackwell, eds., 1986).

Other advantages and characteristics of the invention will become apparent in the examples of implementation of the method of the invention which follow. These examples, which shown that the method of the invention makes it possible to evaluate the immunogenicity, in vitro, of proteins in a specific and sensitive manner, make reference to the attached drawings in which:

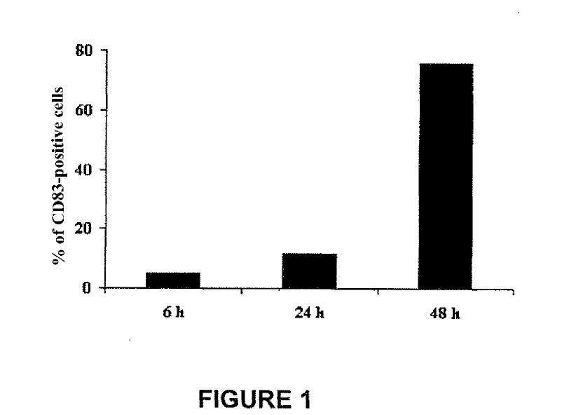

FIG. 1 represents the dendritic cell (DC) maturation kinetics. Immature dendritic cells differentiated from human monocytes were cultured in the presence of lipopolysaccharide (LPS) for 6, 24 or 48 hours. The DC maturation was then evaluated by the expression of the CD83 marker by flow cytometry;

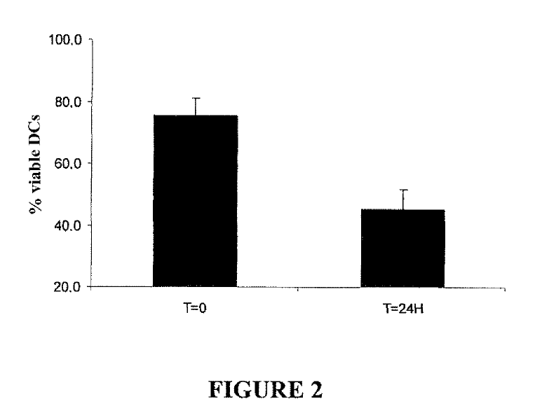

FIG. 2 represents the DC viability kinetics after induction of maturation with LPS. The dendritic cell viability was evaluated by counting the cells after staining with trypan blue. The values correspond to the mean of the results of three donors;

FIG. 3 represents (A) the DC maturation kinetics under various protein-loading and maturation conditions, evaluated by flow cytometry, via the expression of the CD83 marker by the dendritic cells (DC-SIGN positive), and (B) the kinetics of uptake of the protein (OVA-FITC) by the DCs according to various protein-loading and maturation conditions, evaluated by flow cytometry, via the mean fluorescence intensity (MFI) of the OVA-FITC of the dendritic cells (DC-SIGN positive);

FIG. 4 represents the effect of the various modes of OVA loading and of maturation of the DCs on the number of OVA-specific CD4+ T lymphocyte lines. Autologous DCs were prepared either by incubating the immature DCs simultaneously with the OVA and LPS for 24 h, or by incubating the immature DCs with the OVA for 4 h and then with the LPS for 20 h, after an intermediate step of washing the DCs between the loading and the maturation. The OVA-loaded autologous mature DCs thus obtained were used to induce OVA-specific CD4+ T lymphocytes among one donor. The specificity of the OVA-induced CD4+ T lymphocyte lines was evaluated by ELISPOT-IFN-γ;

FIG. 5 represents the effect of the DC OVA-loading and maturation time on the number of OVA-specific CD4+ T lymphocyte lines. Autologous DCs were prepared by incubating the immature DCs simultaneously with OVA and LPS for 24 h or 48 h. The OVA-loaded autologous mature DCs thus obtained were used to induce OVA-specific CD4+ T lymphocytes among one donor. The specificity of the OVA-induced CD4+ T lymphocytes was evaluated by ELISPOT-IFN-γ;

FIG. 6 illustrates the heterogeneity of the responses of CD4+ T lymphocyte lines directed against OVA, evaluated by ELISPOT-IFN-γ. CD4+ T lymphocyte lines directed against OVA were obtained from PBMCs of a single donor and their specificity was tested by ELISPOT-IFN-γ. Each line was tested in triplicate, by ELISPOT-IFN-γ with respect to autologous immature DCs loaded with OVA (black bars), loaded with KLH (hatched bars) and with no loading (white bars). The values correspond to the number of spots per well.

### EXAMPLE 1

### Kinetics of Maturation and Viability of the Dendritic Cells

### 1) Materials and Methods

### a) Preparation of Dendritic Cells

The peripheral blood mononuclear cells (PBMCs) of healthy donors were isolated by density gradient centrifugation (Ficoll-Hypaque gradient, Sigma-Aldrich). The HLA-DR and HLA-DP genotype was determined, either by sequencing of HLA-DRB using suitable PCR primers (Applied Biosystems), or by SSP using the Olerup SSP™ HLA-DPB1 and HLA-DRB1 typing kit (Olerup SSP AB).

The PBMCs were cultured in AIM-V medium (Invitrogen) and incubated in flasks, in an incubator at 37° C. in the presence of 5% CO₂. After incubation overnight, the non-adherent cells (containing the CD4+ T lymphocytes) were recovered. The mononuclear cells adhering to the plastic were differentiated into immature DCs (iDCs) by means of five days of culture in AIM V medium (Invitrogen) supplemented with 1000 U/ml of rh-Gm-CSF (R&D Systems) and 1000 U/ml of rh-IL4 (R&D Systems), hereinafter referred to as complete AIM-V medium. The immature DCs were then differentiated into mature dendritic cells by means of a further incubation in complete AIM-V medium supplemented with 1 μg/ml of LPS.

### b) Flow Cytometry

The DCs were washed twice with FACS buffer (PBS plus 2% FCS) and a double surface labeling was carried out with an anti-DC-SIGN-FITC antibody (BD Biosciences) and an anti-CD83-PE antibody (BD Biosciences), for 20 min at 4° C. After two washes with a buffer for FACS, the cells were fixed with a paraformaldehyde solution (2%; Sigma) for 20 min at 4° C. After two washes with FACS buffer, the cells were resuspended in FACS buffer and analyzed by flow cytometry (FACSCalibur, BD Bioscience).

### c) Cell Viability Analysis

A sample of DCs was taken from the culture, and mixed with a solution of trypan blue which makes it possible to stain the dead cells. The viable (not stained) and nonviable (stained blue) DCs were then counted under an optical microscope, using a hemocytometer (Malassez cell) and the percentage of viable DCs was determined.

### 2) Results

### a) DC Maturation Kinetics

LPS (lipopolysaccharide) is an Escherichia coli cell wall component used to induce DC maturation. The DCs (mature and immature) are detected by the expression of the DC-SIGN marker. On the other hand, unlike immature DCs, mature DCs express a specific marker, which is CD83. It is therefore possible to follow the DC maturation kinetics by flow cytometry, by evaluating the percentage of DC-SIGN-positive cells which express the CD83 marker.

The DC maturation kinetics were evaluated by flow-cytometry analysis of the CD83 labeling, 6 h, 24 h and 48 h after the addition of LPS. The experiment described in FIG. 1 shows that DC maturation is initiated at 24 h at is optimal at 48 h.

### b) DC Viability

The immature DCs generated from monocytes derived from the peripheral blood of healthy donors, as specified above, were cultured at 37° C. for 24 h in complete medium containing 1 μg/ml of LPS, in order to induce maturation thereof.

The viability kinetics of the DCs from three donors, 24 h after the addition of LPS, were evaluated after staining with trypan blue. FIG. 2 shows that the DC viability decreases greatly after 24 h of culture in the presence of LPS.

### EXAMPLE 2

### Protein-Loading and Maturation of DCs

### 1) Materials and Methods

The protein that was used is chicken egg ovalbumin (OVA) labeled with FITC. The immature DCs were prepared and cultured in complete AIM-V medium, as described in example 1. The labeled OVA (150 μg/ml) and the LPS (1 μg/ml) were added to the culture of immature DCs, simultaneously or successively. When the addition is successive, the labeled OVA was added to the culture for 30 min or 2 h and then the cells were washed before adding the LPS. The DCs loaded with labeled OVA were then cultured in the presence of LPS for a total period of 24 h or 48 h. The DC maturation was analyzed by flow cytometry, by double labeling with a fluorescent anti-DC-SIGN antibody and a fluorescent anti-CD83 antibody, as described in example 1. The internalization of the protein by the DCs was analyzed by flow cytometry on fixed cells surface-labelled with a fluorescent anti-DC-SIGN antibody, as described in example 1.

### 2) Results

The ability of the dendritic cells to internalize a protein (FIG. 3A) and their maturation (FIG. 3B) was studied according to various conditions for bringing the cells into contact with a protein and for maturation with LPS.

Three conditions were tested:
OVA+LPS: the protein and the LPS are brought together in contact with the immature DCs for 24 h or 48 h,
OVA 30 min then LPS: the protein is brought into contact with the immature DCs for 30 min, then the cells are washed and incubated with the LPS for the remaining time (23 h30 or 47 h30), and
OVA 2 hours then LPS: the protein is brought into contact with the immature DCs for 2 hours, and then the cell are washed and incubated with the LPS for the remaining time (22 h or 46 h).

It is observed (FIG. 3A) that simultaneously bringing into contact the DCs and the protein does not disturb the maturation of the DCs, whereas the maturation is less efficient at 24 h and 48 h when the antigen is incubated alone with the DCs, which are then washed and put back in culture with the LPS.

In addition, it is observed (FIG. 3B) that simultaneously bringing into contact the DCs and the protein also does not disturb the protein-loading by the DCs. It is more efficient under these conditions than when the protein-uptake phase is separated from the phase of maturation with LPS.

These results therefore show that maturation and protein uptake are more efficient when the protein and the LPS are added to the culture together than when they are added separately.

### EXAMPLE 3

### Comparison of the Ability to Induce CD4+ T Lymphocyte Lines Specific for a Protein According to the DC Loading and Maturation Mode

### 1) Materials and Methods

### a) Obtaining CD4+ T Lymphocyte Lines Specific for the Protein

The immature dendritic cells were obtained from mononuclear cells adhering to the plastic, cultured for 5 days in the presence of IL-4 and of Gm-CSF, as described in example 1. The protein (10 to 500 μg/ml) and the LPS (1 μg/ml) were incubated with the immature DCs simultaneously or successively. When the incubation is successive, the immature DCs and the protein are coincubated in culture medium for 4 h at 37° C. and then the DCs are washed before being incubated with the LPS in culture medium for 20 h or 44 h at 37° C. When the incubation is simultaneous, the immature DCs, the OVA and the LPS are coincubated in culture medium for 24 h or 48 h at 37° C.

The CD4+ T lymphocytes were purified from the non-adherent mononuclear cells of the same donor, by means of magnetic beads to which anti-CD4 antibodies are attached (Miltenyi Biotech). Their purity was verified by flow cytometry. Fifteen wells of 96-well culture plates were seeded with 100 000 CD4+ T lymphocytes and 10 000 mature DCs loaded with the protein, prepared under each of the two conditions specified above, in IMDM medium (Invitrogen) containing 10% of group AB human serum (serum AB), glutamine (24 mM), asparagine (55 mM), arginine (150 mM) (Sigma), 50 U/ml of penicillin and 50 μg/ml of streptomycin (Invitrogen), hereinafter referred to as complete IMDM medium, supplemented with IL-6 (1000 U/ml; R&D Systems) and with IL-12 (10 ng/ml; R&D Systems). After one week and for a further two weeks, the CD4+ T lymphocytes were restimulated with the mature DCs loaded with the protein, prepared under the same conditions as for the initial stimulation, and cultured in complete IMDM medium supplemented with IL-2 (10 U/ml; R&D Systems) and with IL-7 (5 ng/ml; R&D Systems). The specificity of the CD4+ T lymphocyte lines thus obtained was tested by ELISPOT-IFN-γ.

### b) ELISPOT-IFN-γ

An anti-IFN-γ monoclonal antibody (1-D1K, Mabtech, Stockholm, Sweden) diluted to 2.5 μg/ml in PBS (Invitrogen), was adsorbed onto HA multiscreen plates (Millipore) for one hour at 37° C. The plates were then saturated with complete IMDM medium for 1 h.

Autologous immature DCs loaded with the protein were obtained by coincubation of the protein (10 to 500 μg/ml) with autologous immature DCs prepared as described in example 1, at 37° C. for 4 h to 24 h in AIM-V medium, followed by washing of the DCs in 1×PBS and resuspension of the DCs in complete IMDM medium.

Each CD4+ T lymphocyte line (5000 to 20000 cells per well) was cultured with autologous immature DCs (5000 to 20000 cells per well) pre-loaded with the protein or not loaded with the protein. After incubation for 16 to 24 hours at 37° C., the IFN-γ secreted is demonstrated by the successive addition of a biotin-labelled anti-IFN-γ antibody (7-B6-1; Mabtech) (0.25 μg/ml), of streptavidin-phosphatase (Sigma) and of a precipitating substrate NBT/BCIP (Sigma). The presence of IFN-γ-secreting cells is reflected by the appearance of spots at the bottom of the well, each spot corresponding to an IFN-γ-secreting cell. The number of spots per well is evaluated using an Elispot reader (AID). The specific lines produce at least twice as many spots in the presence of DCs loaded with the protein than they do in the presence of non-loaded DCs, with a minimum of 15 spots/10 000 CD4+ T lymphocytes.

### 2) Results

The influence of the loading and maturation load on the ability of the DCs to induce CD4+ T lymphocytes specific for a protein (OVA) was tested (FIG. 4).

OVA-specific CD4+ T lymphocyte lines were induced in vitro by means of weekly stimulations (an initial stimulation followed by three restimulations) with DCs prepared in two different ways:
the immature DCs are brought into contact with the protein (OVA) and LPS for 24 h (OVA+LPS),
the immature DCs are brought into contact with the OVA for 4 h, washed, and incubated with the LPS for 20 h.

The results presented in FIG. 4 shows that loading of the DCs with OVA and simultaneous maturation produce more OVA-specific lines than when the loading and the maturation are carried out one after the other.

### EXAMPLE 4

### Comparison of the Ability to Induce CD4+ T Lymphocyte Lines Specific for a Protein as a Function of the DC Loading and Maturation Time

### 1) Materials and Methods

The protocol for induction and evaluation of the specificity of the CD4+ T lymphocyte lines is the same as that described in example 3.

### 2) Results

In order to optimize the loading and maturation time, OVA-specific CD4+ T lymphocyte lines were induced in vitro by means of weekly stimulations with DCs pre-loaded with OVA and matured simultaneously with LPS for 24 h or 48 h. The results obtained show that the number of OVA-specific CD4+ T lymphocyte lines is greater at 24 h than at 48 h (FIG. 5).

This observation confirms that the optimum conditions for loading and maturation of the DCs in order to induce lines specific for a protein is a balance between, on the one hand, the loading of the protein and the maturation, which both increase with the incubation times (FIG. 1 and FIG. 3) and, on the other hand, the viability of the DCs which, conversely, decreases with the incubation time (FIG. 2).

### EXAMPLE 5

### Demonstration of the Heterogeneity of the Responses of the Lines of CD4+ T Lymphocytes Induced Against a Protein

1) Materials and Methods

Lines of CD4+ T lymphocytes directed against OVA were obtained from the PBMCs of a single donor, in 20 different wells, as described in example 3 and their specificity was evaluated by Elispot as described in example 3.

### 2) Results

The results (FIG. 6) show a high heterogeneity of the values observed, both in the wells where the DCs have been loaded with the protein and when they are not loaded. On the other hand, the triplicates are very homogeneous, demonstrating that the fluctuations observed result from the differences between the CD4+ T lymphocyte lines and not from any lack of precision of the specificity test. Each CD4+ T lymphocyte line behaves independently of the other lines. Although all the wells were seeded under the same conditions, three categories of CD4+ T lymphocyte lines are observed:
1. CD4+ T lymphocyte lines specific for the protein: these lines exhibit more than double the number of spots in the presence of DCs loaded with OVA than they do in the presence of non-loaded DCs, with a minimum number of spots of 15 per 10 000 CD4+ T lymphocytes. This is the case for lines 1 to 9;
2. CD4+ T lymphocyte lines which exhibit a number of spots below the minimum threshold of spots and which are not specific for OVA. These are lines 10 to 17;
3. CD4+ T lymphocyte lines which exceed the minimum number of spots but which exhibit less than double the number of spots in the presence of DCs loaded with OVA than they do in the presence of non-loaded DCs. These CD4+ T lymphocyte lines (18 to 20) are also not specified for OVA, although they give a considerable signal in the presence of DCs loaded with OVA.

Thus, although all the wells were seeded under the same conditions, it is observed that not all the lines are specified for OVA.

### EXAMPLE 6

### Comparison of the Immunogenicity of Immunogenic Proteins and of Nonimmunogenic Proteins

### 1) Materials and Methods

Lines of CD4+ T lymphocytes directed against various proteins which are immunogenic (OVA and KLH) and nonimmunogenic in humans (human insulin, human antitrypsin, human antithrombin) were obtained from PBMCs of three healthy donors and the specificity of each line was evaluated by ELISPOT as described in example 3.

### 2) Results

The method for evaluating immunogenicity was tested on immunogenic proteins (OVA and KLH) and on proteins known not to be immunogenic in humans (human insulin, human antitrypsin, human antithrombin). CD4+ T lymphocyte lines directed against these proteins were obtained from three different donors (Table I).

TABLE 1
Evaluation of the immunogenicity of immunogenic and nonimmunogenic proteins

| | Strength of the response | | | | | |
|---|---|---|---|---|---|---|
| | % | Mean of the | Quotient | Mean of | Mean of | Mean of the |
| | positive | differences | of the | the | the | quotients |
| Protein | wells | (Ai − Bi) | means | values Ai | values Bi | Ai/Bi |
| OVA | 23 | 6.51 | 4.94 | 14 | 7 | 1.78 |
| KLH | 50 | 24.40 | 21.26 | 29 | 4 | 8.15 |
| insulin | 0 | −1.23 | 1.14 | 6 | 7 | 0.84 |
| antitrypsin | 0 | 1.18 | 1.57 | 13 | 11 | 1.10 |
| antithrombin | 3 | 3.20 | 1.22 | 9 | 7 | 1.18 |

For each of the proteins, the mean number of spots per well in the presence of protein-loaded DCs (value Ai) and in the presence of non-loaded DCs (value Bi) was calculated for each of the n T lymphocyte lines (12<n<20) obtained for the three donors (OVA: 55 lines; KLH: 51 lines; insulin: 53 lines; antitrypsin: 51 lines; antithrombin: 40 lines). Each line was tested in triplicate. The results correspond to the means of all the wells tested (three wells per line; OVA: 55 lines; KLH: 51 lines; insulin: 53 lines; antitrypsin: 51 lines; antithrombin: 40 lines).

The percentage of positive wells (2nd column of table I) corresponds to the number of wells comprising a mean number of spots in the presence of protein-loaded DCs (value Ai) which is greater than 15 spots/10 000 lymphocytes and at least double the mean number of spots in the presence of non-loaded DCs (value Bi) over all the wells tested.

The mean of the values Ai (5th column of table I) is calculated from the numbers of spots/well in the presence of protein-loaded DCs (values Ai) measured over all the wells tested.

The mean of the values Bi (6th column of table I) is calculated from the numbers of spots/well in the presence of DCs not loaded with protein (values Bi) measured over all the wells tested.

The quotient of the means (mean of the Ai/mean of the Bi; 4th column of table I) is calculated directly from the above values.

The mean of the differences (3rd column of table I) is calculated from the values (Ai−Bi) measured over all the wells tested.

The mean of the quotients (7th column of table I) is calculated from the quotients (Ai/Bi) measured over all the wells tested.

The results (table I) obtained show that, for KLH and OVA which are immunogenic, the specific-response strengths are high, whereas they are low for the other proteins which are not immunogenic.

### EXAMPLE 7

### Comparison of the Immunogenicity of Therapeutic Proteins

### 1) Materials and Methods

CD4+ T lymphocyte lines directed against various therapeutic proteins (etanercept, infliximab and rituximab) and two reference immunogenic proteins (KLH, murine monoclonal antibody called Mα23) were obtained from PBMCs of four healthy donors and the specificity of each line was evaluated by ELISPOT as described in example 3.

### 2) Results

Etanercept, infliximab and rituximab are therapeutic proteins currently used in human treatments. Their degree of immunogenicity, measured by the percentage of individuals who have specific antibodies, appears to be variable (data published by the European Medicines Agency (EMEA) http://www.emea.europa.eu/ and table II). The immunogenicity of these three proteins was evaluated by the method of the invention on four healthy donors, in comparison with immunogenic proteins (KLH and a murine monoclonal antibody, Mα23).

TABLE II
Evaluation of the immunogenicity of therapeutic proteins: strength of the response

| | | Strength of the response | | | | | Immuno- |
|---|---|---|---|---|---|---|---|
| | | % | Mean of | Mean of | Mean of the | Mean of the | genicity* |
| | | positive | the values | the values | differences | quotients | reported |
| Protein | Type | wells | Ai | Bi | (Ai − Bi) | Ai/Bi | by the EMEA |
| KLH | Nonhuman | 98% | 101 | 7 | 94 | 23.4 | Strong |
| | protein | | | | | | |
| Mα23 | Murine | 48% | 31 | 12 | 19 | 5.1 | strong |
| | monoclonal | | | | | | |
| | antibody | | | | | | |
| Etanercept | Human | 0% | 9 | 8 | 0 | 0.7 | 1-11% |
| | fusion | | | | | | |
| | protein | | | | | | |
| Rituximab | Human- | 7% | 8 | 12 | 0 | 0.8 | 30% |
| | murine | | | | | | |
| | chimeric | | | | | | |
| | antibody | | | | | | |
| Infliximab | Human- | 1% | 14 | 13 | 1 | 1.3 | 13-44% |
| | murine | | | | | | |
| | chimeric | | | | | | |
| | antibody | | | | | | |

*percentage of individuals who have specific antibodies

TABLE III
Evaluation of the immunogenicity of therapeutic proteins:
frequency of responders

| Protein | Frequency of responders (%) |
|---|---|
| KLH | 100% |
| Mα23 | 100% |
| Etanercept | 0% |
| Rituximab | 75% |
| Infliximab | 25% |

The results show that the control proteins (KLH and murine antibodies) which are known to be immunogenic induce high response strengths with a 100% responder frequency. Rituximab which is known to give rise to antibodies in 30% of treated patients, induces few spots, but has a 75% responder frequency. For infliximab, which is known to be immunogenic in 13-44% of patients, the responder frequency and the response strength are further decreased. Finally, etanercept, which is not very immunogenic, does not induce specific CD4+ T lymphocyte lines. The immunogenicity values obtained by means of the method of the invention are of two types. They relate to the strength of the response observed in vitro and to the responder frequency. The responder frequencies are correlated with the values obtained clinically.

### EXAMPLE 8

### Evaluation of the Immunogenicity of a Mixture of Proteins

The method for evaluating immunogenicity according to the invention was tested on a mixture of proteins so as to evaluate whether or not the mixture disturbs the immunogenicity of each protein. An equimolar mixture of KLH, which is very immunogenic, and of human insulin, which is not, was incubated with autologous dendritic cells in order to stimulate CD4+ T lymphocytes, according to the protocol described in example 3. The T lymphocyte lines thus obtained were tested by IFN-γ ELISpot with respect to dendritic cells alone, to dendritic cells loaded with KLH and to dendritic cells loaded with human insulin, according to the protocol described in example 3. The experiment was carried out on four different donors. The immunogenicity of each of the proteins of the mixture was calculated as described in example 6.

TABLE IV
Evaluation of the immunogenicity in vitro of therapeutic
proteins by means of a mixture of proteins

| | | Mean of spots | | | | |
|---|---|---|---|---|---|---|
| | | Mean of the | Quotient | Mean of | Mean of | Mean of the |
| | Positive | differences | of the | the | the | quotients |
| Protein | wells (%) | (Ai − Bi) | means Ai/Bi | values Ai | values Bi | Ai/Bi |
| KLH | 94 | 106 | 4 | 132 | 31 | 13 |
| insulin | 0 | −2 | 1 | 26 | 31 | 1 |

Values A (+protein)
Values B (no protein)

The results obtained show that KLH induces high response strengths (table IV) with a 100% responder frequency. Insulin does not give any specific response in any of the donors. The results obtained are therefore highly comparable to those obtained with the two proteins separately. This therefore shows that the immunogenicity of different proteins can be tested although these proteins are mixed.

### EXAMPLE 9

### Evaluation of the Immunogenicity of a Human Antibody

This additional study was carried out on a human antibody (Adalimumab) known to be possibly immunogenic in humans. Indeed, the specific antibody responses observed in individuals treated with Adalimumab can involve up to 87% of patients (table V).

CD4+ T lymphocyte lines directed against Adalimumab and against a reference protein (KLH) were obtained from PBMCs from nine healthy donors and the specificity of each line was evaluated by IFN-γ Elispot as described in the previous examples.

TABLE V
Evaluation of the immunogenicity of therapeutic proteins: strength of the response

| | | | Mean of spots | | | | | Immuno- |
|---|---|---|---|---|---|---|---|---|
| | | | Mean of the | Quotient | Mean of | Mean of | Mean of | genicity |
| | | Positive | differ- | of the | the | the | the | reported |
| | | wells | ences | means | values | values | quotients | in clinical |
| Protein | Type | (%) | (Ai − Bi) | Ai/Bi | Ai | Bi | Ai/Bi | studies |
| KLH | Nonhuman | 88 | 215 | 3 | 311 | 97 | 3.64 | strong |
| | protein | | | | | | | |
| Adalimumab | Human | 13 | −6 | 1 | 85 | 90 | 1.08 | 4 to 87% |
| | antibody | | | | | | | |

TABLE VI
Evaluation of the immunogenicity of therapeutic proteins:
responder frequency

| Protein | Type | Responder frequency |
|---|---|---|
| KLH | Nonhuman | 100% |
| adalimumab | Human | 67% |

These results show that there is a strong response against the reference protein (KLH). The response strength measured by the percentage of positive wells or by the number of spots (values A) relative to the control without protein (values B) is very high. All the donors are responders.

The Adalimumab human antibody induces a response of low strength but which is observed in 67% of donors. This percentage of responders is in agreement with the observations made clinically.

All these results show that the method of the invention makes it possible to evaluate the immunogenicity of therapeutic proteins and that it provides information on the risks of responses in humans:

Specifically:
a high strength and a high responder frequency reflect a high risk of strong immune response;
a low strength and a high frequency reflect a high risk of response, but of lower strength;
a high strength and a low frequency reflect a high risk of response for a limited number of individuals. This is a population at risk;
a low strength and a low responder frequency reflect a low risk of immunogenicity.

### EXAMPLE 10

### Evaluation of the Frequency of Lymphocytes Specific to Therapeutic Proteins

The method of the invention also makes it possible to evaluate the frequency of CD4+ T lymphocytes specific for the therapeutic protein among the CD4+ T lymphocytes of the donor.

Specifically, the results obtained show that, among all the wells that were stimulated with the protein, there are wells which do not give rise to CD4+ T lymphocyte lines specific to the protein. This is explained by the fact that, during the distribution of the CD4+ T lymphocytes derived from the donor, in the culture wells, these wells have not received specific CD4+ T lymphocytes. The frequency of these specific CD4+ T lymphocytes is very low, to the extent that less than one cell per well is distributed. The distribution follows Poisson's law, also known as the law of rare events.

Very specifically, the distribution of the specific CD4+ T lymphocytes follows a Poisson's law with parameter λ, where λ is the mean number of specific CD4+ T lymphocytes per well.

The probability P(X=k) of there being k specific CD4+ T lymphocytes in a well is given by the formula: P(X=k)=e^{-λ}λ^{k}/k!.

It is then possible to calculate the probability that a well does not contain specific cells by taking the value k=0, this value is P(X=0)=e^{-λ}.

The value of λ=ln(P(X=0)), which depends on the number of negative wells, is deduced therefrom. The number of negative wells is in fact an estimation of P(X=0). This number of negative wells can be easily calculated. The number of specific CD4+ T lymphocytes per number of CD4+ T lymphocytes distributed is then obtained from the number of CD4+ T lymphocytes distributed per well and from the value calculated for λ. This calculation was performed for several of the proteins previously studied (table VII). The mean frequency is expressed as number of specific CD4+ T lymphocytes per million CD4+ T lymphocytes.

TABLE VII
Examples of frequency of CD4+ T lymphocytes specific
for therapeutic proteins

| Protein | Frequency of CD4+ T lymphocytes |
|---|---|
| KLH | 15.98 |
| Mα23 | 2.26 |
| Rituximab | 0.41 |
| Adalimumab | 0.44 |

1. A method for evaluating the immunogenicity of proteins, comprising at least the following steps:
a) activating CD4+ T lymphocytes of at least one donor by coculturing CD4+ T lymphocytes of each donor with autologous antigen-presenting cells loaded with a test protein, in at least ten independent culture vessels (n≧10) for each donor,
b) measuring the activation of said CD4+ T lymphocytes with respect to autologous immature dendritic cells loaded with said protein (value Ai with 1≦i≦n) and measuring, in parallel, the activation of said CD4+ T lymphocytes with respect to non-loaded autologous immature dendritic cells (value Bi with 1≦i≦n), for each of the n cultures of CD4+ T lymphocytes of each donor of step a), and
c) calculating the value of the immunogenicity of said protein by comparison of all the values Ai with all the values Bi, obtained for all the cultures of CD4+ T lymphocytes of the donors of step a).

2. The method as claimed in claim 1, wherein the antigen-presenting cells loaded with the test protein (step a)) are mature dendritic cells loaded with said protein.

3. The method as claimed in claim 2, wherein said mature dendritic cells have been obtained by maturation of immature dendritic cells in the presence of LPS.

4. The method as claimed in claim 2, wherein said mature dendritic cells have been obtained from immature dendritic cells which have been loaded with the protein and matured simultaneously with the maturation agent.

5. The method as claimed in claim 4, wherein said immature dendritic cells have been incubated simultaneously with the protein and the maturation agent for at least 4 hours.

6. The method as claimed in claim 1, wherein step a) comprises at least one restimulation of the CD4+ T lymphocytes by addition, to the coculture, of autologous antigen-presenting cells loaded with said test protein.

7. The method as claimed in claim 6, wherein the restimulations are carried out every 5 to 7 days, the first being carried out after at least 5 days of coculture.

8. The method as claimed in claim 6, wherein it comprises two restimulations 5 to 7 days apart, the first being carried out after 5 to 7 days of coculture.

9. The method as claimed in claim 1, wherein the measurement of the activation of the CD4+ T lymphocytes comprises measuring the proliferation of said CD4+ T lymphocytes or else measuring the production of cytokine(s) or the expression of activation marker(s) by said CD4+ T lymphocytes.

10. The method as claimed in claim 9, wherein the activation of said CD4+ T lymphocytes is measured by means of a lymphocyte proliferation assay, an intracellular cytokine(s) labeling assay or an ELISPOT assay.

11. The method as claimed in claim 1, wherein the calculation of the value of the immunogenicity of the protein (step c)) comprises calculating the strength of the specific CD4+ T response.

12. The method as claimed in claim 11, wherein the strength of the specific CD4+ T response is expressed by the frequency of positive culture vessels, corresponding to those of which the value Ai is greater than the background noise and at least double the value Bi.

13. The method as claimed in claim 11, wherein the strength of the specific CD4+ T response is expressed by the mean of the differences between the values Ai and Bi, the mean of the quotients Ai over Bi or the quotient of the mean of the values Ai over the mean of the values Bi.

14. The method as claimed in claim 13, wherein the strength is significant when the mean of the differences between the values Ai and Bi is greater than the background noise, or else the mean of the quotients Ai over Bi or the quotient of the mean of the values Ai over the mean of the values Bi is greater than or equal to 2.

15. The method as claimed in claim 11, wherein the strength of the specific CD4+ T response is expressed by the frequency of CD4+ T lymphocytes specific for said protein among the CD4+ T lymphocytes of the donor.

16. The method as claimed in claim 1, wherein said protein is a therapeutic protein.

17. The method as claimed in claim 1, wherein said protein is included in a mixture of different proteins.

18. The method as claimed in claim 1, wherein said donor is naive with respect to said protein.

19. The method as claimed in claim 1, wherein steps a) to c) are carried out in parallel on cultures derived from a collection of donors, step c) comprising calculating the strength of the CD4+ T response and the frequency of responder individuals in the collection of donors.

20. The method as claimed in claim 19, wherein steps a) to c) are carried out in parallel on cultures derived from at least three different donors.

21. The method as claimed in claim 19, the frequencies of the HLA-DR alleles in the collection of donors are close to those encountered in the population to be studied.

22. The method as claimed in claim 1, wherein, for comparing the immunogenicity of several proteins, steps a) to c) are carried out successively or simultaneously with the various test proteins and then the immunogenicity values obtained in step c) are compared with one another.

23. The method as claimed in claim 22, wherein it comprises comparing the immunogenicity of at least one test protein with that of a reference protein.

24. The method as claimed in claim 22, characterized in that it comprises comparing the immunogenicity of similar proteins, in particular of variants of a protein which have been improved by directed evolution.

25. The method as claimed in claim 22, wherein it comprises comparing the immunogenicity of at least one modified protein with that of an unmodified reference protein.

26. The method as claimed in claim 21, wherein it comprises comparing the immunogenicity of the same protein at various steps of its production process, according to its production batch, according to its formulation or its method of production.

27. The use of the method as claimed in claim 1, for screening for and selecting therapeutic proteins having a desired immunogenicity.

28. The use of the method as claimed in claim 1, for testing the immunogenicity of a therapeutic protein during its production or its formulation.

29. The use of the method as claimed in claim 1, for comparing the immunogenicity of a therapeutic protein in various populations of individuals.
